(19) United States
(12) Patent Application Publication
(10) Pub. No.: US 2012/0215295 A1
(43) Pub. Date: Aug. 23, 2012

(54) SYSTEMS AND METHODS FOR CUSTOMIZING ELECTRODE STIMULATION

(75) Inventor: Anne Margaret Pianca, Santa Monica, CA (US)

(73) Assignee: Boston Scientific Neuromodulation Corporation, Valencia, CA (US)

(21) Appl. No.: 13/370,046

(22) Filed: Feb. 9, 2012

Related U.S. Application Data
(60) Provisional application No. 61/444,061, filed on Feb. 17, 2011.

(51) Int. Cl. A61N 1/04

(52) U.S. Cl. 607/116

(57) ABSTRACT

A lead assembly for an electrical stimulation system includes a lead scaffold that defines first, second, and third channels defined along the first major surface of the lead scaffold. The first, second, and third channels are parallel to one another and to a longitudinal length of the lead scaffold. A tapered guide feature is coupled to one end of the lead scaffold. The lead assembly also includes first and second leads with electrodes at distal ends of the leads, terminals at proximal ends of the leads, and conductive wires coupling the electrodes to the terminals. The first lead is insertable into the first channel and the second lead is insertable into the second channel.

### CROSS-REFERENCE TO RELATED APPLICATIONS

This application claims the benefit under 35 U.S.C. §119(e) of U.S. Provisional Patent Application Ser. No. 61/444,061 filed on Feb. 17, 2011, which is incorporated herein by reference.

### FIELD

The present invention is directed to the area of implantable electrical stimulation systems and methods of making and using the systems. The present invention is also directed to implantable electrical stimulation leads with customizable electrode configurations, as well as methods of making and using the electrodes, leads, and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat incontinence, as well as a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

In one embodiment, a lead assembly for an electrical stimulation system includes a substantially planar lead scaffold having a first end, an opposing second end, a longitudinal length, a first major surface, and an opposing second major surface. The lead scaffold defines a first channel, a second channel, and a third channel each defined along the first major surface from the first end of the lead scaffold. The first, second, and third channels are parallel to one another and to the longitudinal length of the lead scaffold. A guide feature can be coupled to the first end of the lead scaffold. The guide feature has a connection end, an opposing guiding end, and a longitudinal length. The connection end is coupled to the lead scaffold. The guide feature is tapered along its longitudinal length such that the connection end has a diameter that is greater than a diameter of the guiding end. The lead assembly also includes a first lead and a second lead. The first and second leads each have a proximal end and an opposing distal end. For each of the first and second leads a plurality of electrodes are disposed at the distal end of the lead, a plurality of terminals disposed at the proximal end of the lead, and a plurality of conductive wires couple the plurality of electrodes electrically to the plurality of terminals. The first lead is insertable into the first channel such that at least one of the electrodes of the first lead is disposed in the first channel between the first end and the second end of the lead scaffold. The second lead is insertable into the second channel such that at least one of the electrodes of the second lead is disposed in the second channel between the first end and the second end of the lead scaffold.

In another embodiment, an electrical stimulation kit includes a plurality of connectable stimulation members configured and arranged for coupling together with one another. Each of the connectable stimulation members includes a member body and at least one electrode disposed on the member body. Each of the connectable stimulation members also includes a mechanical coupling element disposed on an edge of the member body. The mechanical coupling element is configured and arranged to mechanically couple with a corresponding mechanical coupling element of another one of the plurality of connectable stimulation members. Each of the connectable stimulation members further includes at least one lead body coupled to the member body. At least one terminal is disposed on each of the at least one lead bodies. For each of the connectable stimulation members conductive wires couple the at least one electrode electrically to the at least one terminal.

In yet another embodiment, a lead for an electrical stimulation system includes a plurality of connectable stimulation members coupled together with one another. Each of the connectable stimulation members includes a member body and at least one electrode disposed on the member body. Each of the connectable stimulation members also includes at least mechanical coupling element disposed on an edge of the paddle body. The at least one coupling element may be configured and arranged to mechanically couple with a corresponding mechanical coupling element of another one of the connectable stimulation members. Each of the connectable stimulation members further includes at least one lead body coupled to the member body. At least one terminal is disposed on each of the at least one lead bodies. For each of the connectable stimulation members conductive wires couple the at least one electrode electrically to the at least one terminal.

In another embodiment, a method of customizing stimulation by an implantable stimulation system includes providing a substantially planar lead scaffold having a first end, an opposing second end, a longitudinal length, a first major surface, and an opposing second major surface. The lead scaffold defines a first channel, a second channel, and a third channel each defined along the first major surface from the first end of the lead scaffold. The first, second, and third channels are each parallel to one another and extend along axes parallel to the longitudinal length. A guide feature is coupled to the first end of the lead scaffold. The guide feature has a connection end, an opposing guiding end, and a longitudinal length. The connection end is coupled to the lead scaffold. The guide feature is tapered along its longitudinal length such that the connection end has a diameter that is greater than a diameter of the guiding end. A first lead is inserted into the first channel of the lead scaffold. The first lead has a proximal end and an opposing distal end. The first lead includes a plurality of electrodes disposed at the distal end of the first lead, a plurality of terminals disposed at the proximal end of the first lead, and a plurality of conductive wires coupling the plurality of electrodes electrically to the plurality of terminals. A second lead is inserted into the second channel of the lead scaffold. The second lead has a proximal end and an opposing distal end. The second lead includes a plurality of electrodes disposed at the distal end of the second lead, a plurality of terminals disposed at the proximal end of the second lead, and a plurality of conductive wires coupling the plurality of electrodes electrically to the plurality of terminals. The lead scaffold is implanted into a patient.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes a paddle body coupled to a control module via lead bodies, according to the invention;

FIG. 2 is a schematic view of another embodiment of an electrical stimulation system that includes a percutaneous lead body coupled to a control module via a lead body, according to the invention;

FIG. 3A is a schematic view of one embodiment of a plurality of connector assemblies disposed in the control module of FIG. 1, the connector assemblies configured and arranged to receive the proximal portions of the lead bodies of FIG. 1, according to the invention;

FIG. 3B is a schematic view of one embodiment of a connector assembly disposed in the control module of FIG. 2, the connector assembly configured and arranged to receive the proximal portion of one of the lead body of FIG. 2, according to the invention;

FIG. 3C is a schematic view of one embodiment of a proximal portion of the lead body of FIG. 2, a lead extension, and the control module of FIG. 2, the lead extension configured and arranged to couple the lead body to the control module, according to the invention;

FIG. 4A is a schematic top view of one embodiment of the paddle body of FIG. 1 having a 2×8 electrode configuration, according to the invention;

FIG. 4B is a schematic top view of one embodiment of a paddle body having a 1×8 electrode configuration, according to the invention;

FIG. 4C is a schematic top view of one embodiment of a paddle body having a 3×8 electrode configuration, according to the invention;

FIG. 4D is a schematic top view of one embodiment of a paddle body having a 4×8 electrode configuration, according to the invention;

FIG. 4E is a schematic top view of one embodiment of a paddle body having a 5×8 electrode configuration, according to the invention;

FIG. 5A is a schematic top view of one embodiment of a lead scaffold defining channels for receiving one or more of the paddle bodies of FIG. 4B, one or more of the percutaneous leads of FIG. 2, or both, according to the invention;

FIG. 5B is a schematic side view of one embodiment of the lead scaffold of FIG. 5A, the lead scaffold defining channels for receiving one or more of the paddle bodies of FIG. 4B, according to the invention;

FIG. 5C is a schematic side view of one embodiment of the lead scaffold of FIG. 5A, the lead scaffold defining channels for receiving one or more of the percutaneous leads of FIG. 2, according to the invention;

FIG. 6 is a schematic top view of another embodiment of a lead scaffold defining channels for receiving the one or more of the paddle leads of FIG. 4B, one or more of the percutaneous leads of FIG. 2, or both, according to the invention;

FIG. 7 is a schematic top view of yet another embodiment of a lead scaffold defining channels for receiving one or more of the paddle leads of FIG. 4B, one or more of the percutaneous leads of FIG. 2, or both, according to the invention;

FIG. 8A is a schematic top view of another embodiment of a lead scaffold defining channels for receiving one or more of the paddle bodies of FIG. 4B, one or more of the percutaneous leads of FIG. 2, or both, the lead scaffold including a guide tube, according to the invention;

FIG. 8B is a schematic side view of one embodiment of the lead scaffold and guide tube of FIG. 8A, according to the invention;

FIG. 9A is a schematic top view of one embodiment of a plurality of the paddle bodies of FIG. 4B disposed on the lead scaffold of FIG. 5A, according to the invention;

FIG. 9B is a schematic top view of another embodiment of a plurality of the paddle bodies of FIG. 4B disposed on the lead scaffold of FIG. 5A, according to the invention;

FIG. 9C is a schematic top view of yet another embodiment of a plurality of the paddle bodies of FIG. 4B disposed on the lead scaffold of FIG. 5A, according to the invention;

FIG. 10 is a schematic top view of one embodiment of a plurality of the paddle bodies of FIG. 4B disposed on the lead scaffold of FIG. 8, according to the invention;

FIG. 11A is a schematic top view of one embodiment of a plurality of connectable stimulation members each having 2×4 electrode configurations, the connectable stimulation members combinable to form the paddle body of FIG. 4A with a 2×8 electrode configuration, according to the invention;

FIG. 11B is a schematic top view of one embodiment of a plurality of connectable stimulation members each having 2×2 electrode configurations, the connectable stimulation members combinable to form the paddle body of FIG. 4A with a 2×8 electrode configuration, according to the invention;

FIG. 12A is a schematic top view of one embodiment of a plurality of connectable stimulation members each having 1×8 electrode configurations, the connectable stimulation members combinable to form the paddle body of FIG. 4A with a 2×8 electrode configuration, according to the invention;

FIG. 12B is a schematic top view of one embodiment of a plurality of connectable stimulation members with 2×8 electrode configurations, the connectable stimulation members combinable to form the paddle body of FIG. 4D with a 4×8 electrode configuration, according to the invention;

FIG. 12C is a schematic top view of one embodiment of a plurality of connectable stimulation members with 1×8 electrode configurations, the connectable stimulation members combinable to form the paddle body of FIG. 4D with a 4×8 electrode configuration, according to the invention;

FIG. 13A is a schematic top view of one embodiment of a plurality of connectable stimulation members each having 1×4 electrode configurations, the connectable stimulation members combinable to form the paddle body of FIG. 4A with a 2×8 electrode configuration, according to the invention;

FIG. 13B is a schematic top view of one embodiment of a plurality of connectable stimulation members each having 2×4 electrode configurations, the connectable stimulation members combinable to form the paddle body of FIG. 4D with a 4×8 electrode configuration, according to the invention;

FIG. 14 is a schematic top view of one embodiment of a plurality of connectable stimulation members each having 2×4 electrode configurations and a spacer therebetween, the connectable stimulation members and the spacer combinable to form a paddle body with a 2×8 electrode configuration, according to the invention; and

FIG. 15 is a schematic overview of one embodiment of components of a stimulation system, including an electronic subassembly disposed within a control module, according to the invention.

### DETAILED DESCRIPTION

The present invention is directed to the area of implantable electrical stimulation systems and methods of making and using the systems. The present invention is also directed to implantable electrical stimulation leads with customizable electrode configurations, as well as methods of making and using the electrodes, leads, and electrical stimulation systems.

Suitable implantable electrical stimulation systems include, but are not limited to, an electrode lead (“lead”) with one or more electrodes disposed on a distal end of the lead and one or more terminals disposed on one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,244,150; 7,672,734; 7,761,165; 7,949,395; and 7,974,706; and U.S. Patent Applications Publication Nos. 2005/0165465, 2007/0150036; 2007/0219595; and 2008/0071320, all of which are incorporated by reference.

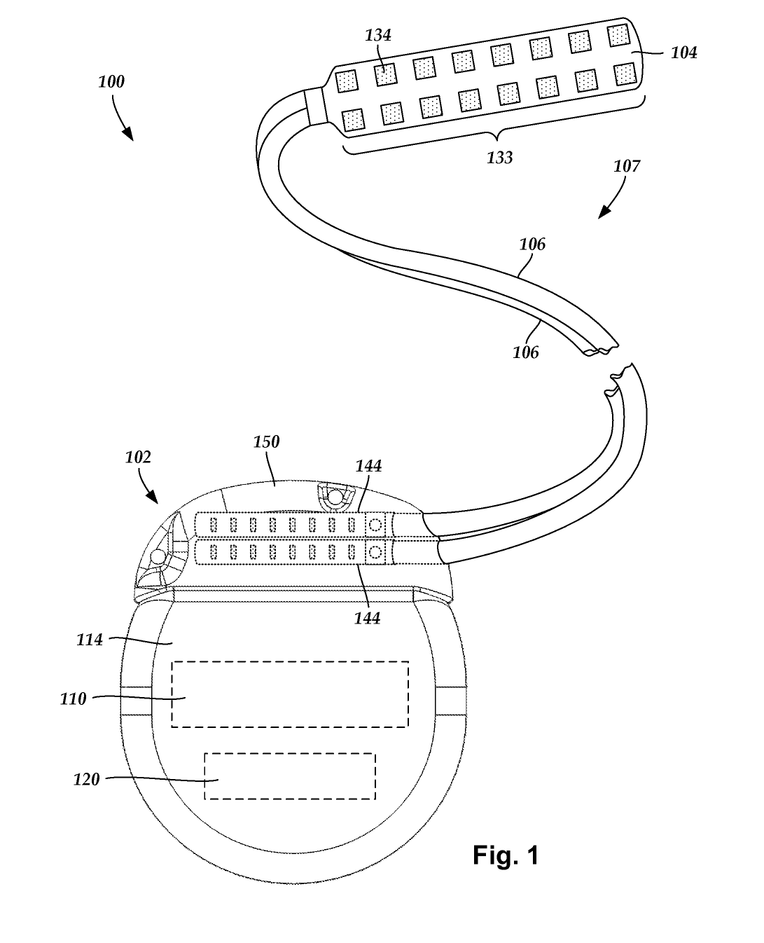

FIG. 1 illustrates schematically one embodiment of an electrical stimulation system 100. The electrical stimulation system includes a control module (e.g., a stimulator or pulse generator) 102, a paddle body 104, and one or more lead bodies 106 coupling the control module 102 to the paddle body 104. The paddle body 104 and the one or more lead bodies 106 form a paddle lead 107. The paddle body 104 typically includes a plurality of electrodes 134 that form an array of electrodes 133. The control module 102 typically includes an electronic subassembly 110 and an optional power source 120 disposed in a sealed housing 114. In FIG. 1, two lead bodies 106 are shown coupled to the control module 102.

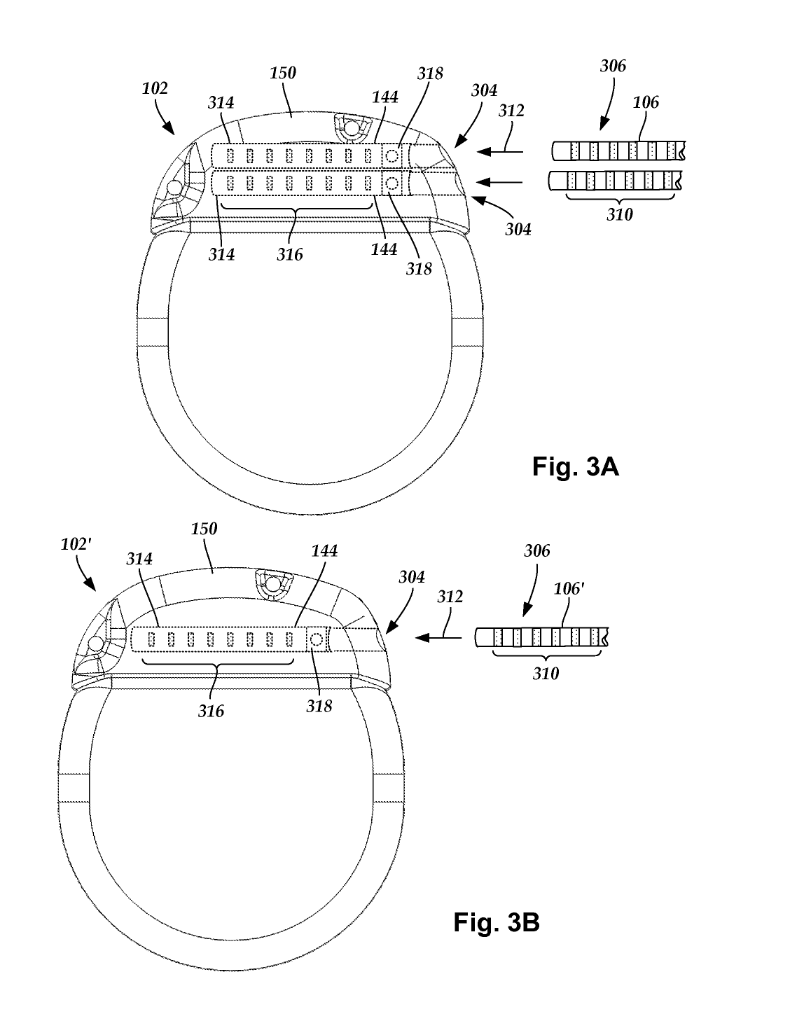

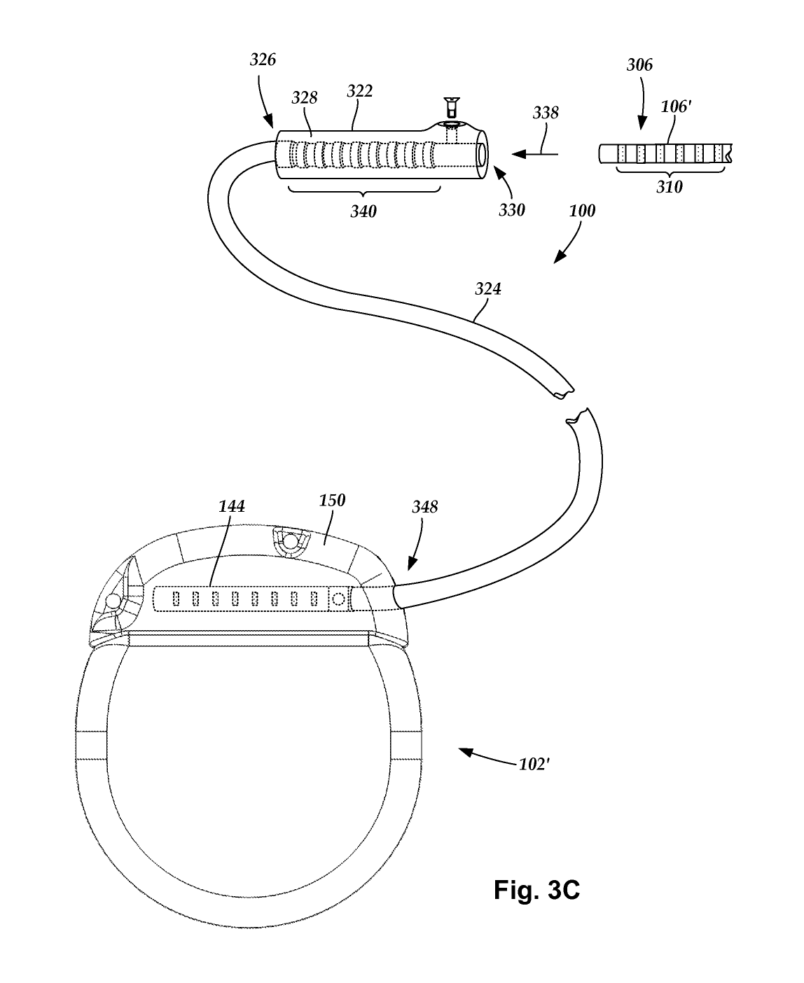

The control module 102 typically includes one or more connector assemblies 144 into which the proximal end of the one or more lead bodies 106 can be plugged to make an electrical connection via connector contacts (e.g., 316 in FIG. 3A) disposed in the connector assembly 144 and terminals (e.g., 310 in FIG. 3A) on each of the one or more lead bodies 106. The connector contacts are coupled to the electronic subassembly 110 and the terminals are coupled to the electrodes 134. In FIG. 1, two connector assemblies 144 are shown.

The one or more connector assemblies 144 may be disposed in a header 150. The header 150 provides a protective covering over the one or more connector assemblies 144. The header 150 may be formed using any suitable process including, for example, casting, molding (including injection molding), and the like. In addition, one or more lead extensions 324 (see FIG. 3C) can be disposed between the one or more lead bodies 106 and the control module 102 to extend the distance between the one or more lead bodies 106 and the control module 102.

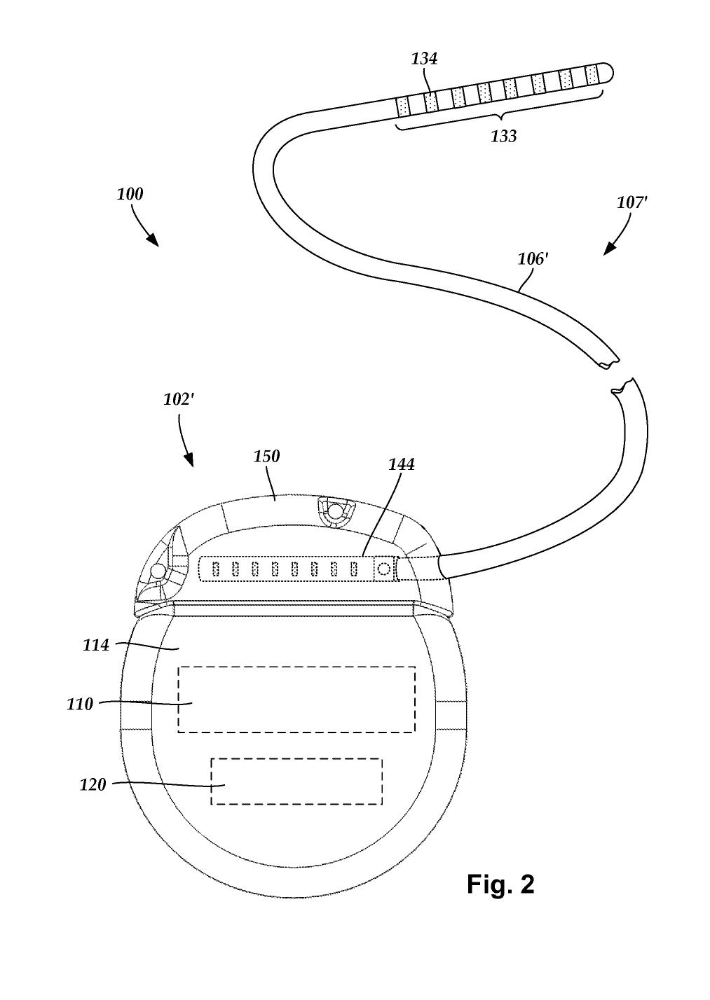

It will be understood that the electrical stimulation system can include more, fewer, or different components and can have a variety of different configurations including those configurations disclosed in the electrical stimulation system references cited herein. For example, instead of a paddle body 104, the electrodes 134 can be disposed in an array at or near the distal end of a lead body 106′ forming a percutaneous lead 107′, as illustrated in FIG. 2. The percutaneous lead may be isodiametric along the length of the lead body 106″. The lead body 106′ can be coupled with a control module 102′ with a single connector assembly 144.

The electrical stimulation system or components of the electrical stimulation system, including one or more of the lead bodies 106, the control module 102, and, in the case of the paddle lead 107, the paddle body 104, are typically implanted into the body of a patient. The electrical stimulation system can be used for a variety of applications including, but not limited to, spinal cord stimulation, brain stimulation, neural stimulation, muscle activation via stimulation of nerves innervating muscle, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium.

The number of electrodes 134 in the array of electrodes 133 may vary. For example, there can be two, three, four, five, six, seven, eight, nine, ten, eleven, twelve, thirteen, fourteen, fifteen, sixteen, or more electrodes 134. As will be recognized, other numbers of electrodes 134 may also be used. In FIG. 1, sixteen electrodes 134 are shown. The electrodes 134 can be formed in any suitable shape including, for example, round, oval, triangular, rectangular, pentagonal, hexagonal, heptagonal, octagonal, or the like.

The electrodes of the paddle body 104 or one or more lead bodies 106 are typically disposed in, or separated by, a non-conductive, biocompatible material including, for example, silicone, polyurethane, and the like or combinations thereof. The paddle body 104 and one or more lead bodies 106 may be formed in the desired shape by any process including, for example, molding (including injection molding), casting, and the like. Electrodes and connecting wires can be disposed onto or within a paddle body either prior to or subsequent to a molding or casting process. The non-conductive material typically extends from the distal end of the lead to the proximal end of each of the one or more lead bodies 106. The non-conductive, biocompatible material of the paddle body 104 and the one or more lead bodies 106 may be the same or different. The paddle body 104 and the one or more lead bodies 106 may be a unitary structure or can be formed as two separate structures that are permanently or detachably coupled together.

Terminals (e.g., 310 in FIG. 3A) are typically disposed at the proximal end of the one or more lead bodies 106 for connection to corresponding conductive contacts (e.g., 316 in FIG. 3A) in connector assemblies (e.g., 144 in FIG. 1) disposed on, for example, the control module 102 (or to other devices, such as conductive contacts on a lead extension, an operating room cable, a splitter, an adaptor, or the like).

Conductive wires (not shown) extend from the terminals (e.g., 310 in FIG. 3A) to the electrodes 134. Typically, one or more electrodes 134 are electrically coupled to a terminal (e.g., 310 in FIG. 3A). In some embodiments, each terminal (e.g., 310 in FIG. 3A) is only coupled to one electrode 134.

The conductive wires may be embedded in the non-conductive material of the lead or can be disposed in one or more lumens (not shown) extending along the lead. In some embodiments, there is an individual lumen for each conductive wire. In other embodiments, two or more conductive wires may extend through a lumen. There may also be one or more lumens (not shown) that open at, or near, the proximal end of the lead, for example, for inserting a stylet wire to facilitate placement of the lead within a body of a patient. Additionally, there may also be one or more lumens (not shown) that open at, or near, the distal end of the lead, for example, for infusion of drugs or medication into the site of implantation of the paddle body 104. The one or more lumens may, optionally, be flushed continually, or on a regular basis, with saline, epidural fluid, or the like. The one or more lumens can be permanently or removably sealable at the distal end.

As discussed above, the one or more lead bodies 106 may be coupled to the one or more connector assemblies 144 disposed on the control module 102. The control module 102 can include any suitable number of connector assemblies 144 including, for example, two three, four, five, six, seven, eight, or more connector assemblies 144. It will be understood that other numbers of connector assemblies 144 may be used instead. In FIG. 1, each of the two lead bodies 106 includes eight terminals that are shown coupled with eight conductive contacts disposed in a different one of two different connector assemblies 144.

FIG. 3A is a schematic side view of one embodiment of a plurality of connector assemblies 144 disposed on the control module 102. In at least some embodiments, the control module 102 includes two connector assemblies 144. In at least some embodiments, the control module 102 includes four connector assemblies 144. In FIG. 3A, proximal ends 306 of the plurality of lead bodies 106 are shown configured and arranged for insertion to the control module 102. FIG. 3B is a schematic side view of one embodiment of a single connector assembly 144 disposed on the control module 102′. In FIG. 3B, the proximal end 306 of the single lead body 106′ is shown configured and arranged for insertion to the control module 102′.

In FIGS. 3A and 3B, the one or more connector assemblies 144 are disposed in the header 150. In at least some embodiments, the header 150 defines one or more ports 304 into which the proximal end(s) 306 of the one or more lead bodies 106/106′ with terminals 310 can be inserted, as shown by directional arrows 312, in order to gain access to the connector contacts disposed in the one or more connector assemblies 144.

The one or more connector assemblies 144 each include a connector housing 314 and a plurality of connector contacts 316 disposed therein. Typically, the connector housing 314 defines a port (not shown) that provides access to the plurality of connector contacts 316. In at least some embodiments, one or more of the connector assemblies 144 further includes a retaining element 318 configured and arranged to fasten the corresponding lead body 106/106′ to the connector assembly 144 when the lead body 106/106′ is inserted into the connector assembly 144 to prevent undesired detachment of the lead body 106/106′ from the connector assembly 144. For example, the retaining element 318 may include an aperture through which a fastener (e.g., a set screw, pin, or the like) may be inserted and secured against an inserted lead body 106/106′.

When the one or more lead bodies 106/106′ are inserted into the one or more ports 304, the connector contacts 316 can be aligned with the terminals 310 disposed on the one or more lead bodies 106/106′ to electrically couple the control module 102 to the electrodes (134 of FIG. 1) disposed at a distal end of the one or more lead bodies 106. Examples of connector assemblies in control modules are found in, for example, U.S. Pat. No. 7,244,150 and U.S. Patent Application Publication No. 2008/0071320, which are incorporated by reference.

In at least some embodiments, the electrical stimulation system includes one or more lead extensions. The one or more lead bodies 106/106′ can be coupled to one or more lead extensions which, in turn, are coupled to the control module 102/102′. In FIG. 3C, a lead extension connector assembly 322 is disposed on a lead extension 324. The lead extension connector assembly 322 is shown disposed at a distal end 326 of the lead extension 324. The lead extension connector assembly 322 includes a contact housing 328. The contact housing 328 defines at least one port 330 into which a proximal end 306 of the lead body 106′ with terminals 310 can be inserted, as shown by directional arrow 338. The lead extension connector assembly 322 also includes a plurality of connector contacts 340. When the lead body 106′ is inserted into the port 330, the connector contacts 340 disposed in the contact housing 328 can be aligned with the terminals 310 on the lead body 106 to electrically couple the lead extension 324 to the electrodes (134 of FIG. 1) disposed at a distal end (not shown) of the lead body 106′.

The proximal end of a lead extension can be similarly configured and arranged as a proximal end of a lead body. The lead extension 324 may include a plurality of conductive wires (not shown) that electrically couple the connector contacts 340 to terminal on a proximal end 348 of the lead extension 324. The conductive wires disposed in the lead extension 324 can be electrically coupled to a plurality of terminals (not shown) disposed on the proximal end 348 of the lead extension 324. In at least some embodiments, the proximal end 348 of the lead extension 324 is configured and arranged for insertion into a lead extension connector assembly disposed in another lead extension. In other embodiments (as shown in FIG. 3C), the proximal end 348 of the lead extension 324 is configured and arranged for insertion into the connector assembly 144 disposed on the control module 102′.

It will be understood that the control modules 102/102′ can receive either lead bodies 106/106′ or lead extensions 324. It will also be understood that the electrical stimulation system 100 can include a plurality of lead extensions 224. For example, each of the lead bodies 106 shown in FIGS. 1 and 3A can, alternatively, be coupled to a different lead extension 224 which, in turn, are each coupled to different ports of a two-port control module, such as the control module 102 of FIGS. 1 and 3A.

In the case of paddle leads 107, electrodes 134 can be disposed on the paddle body 104 in any suitable arrangement. For example, in FIG. 1 the electrodes 134 are shown in a configuration that includes rows and columns. In FIG. 1, the paddle body 104 is shown having two electrodes 134 per row and eight electrodes 134 per column, or a “2×8” configuration.

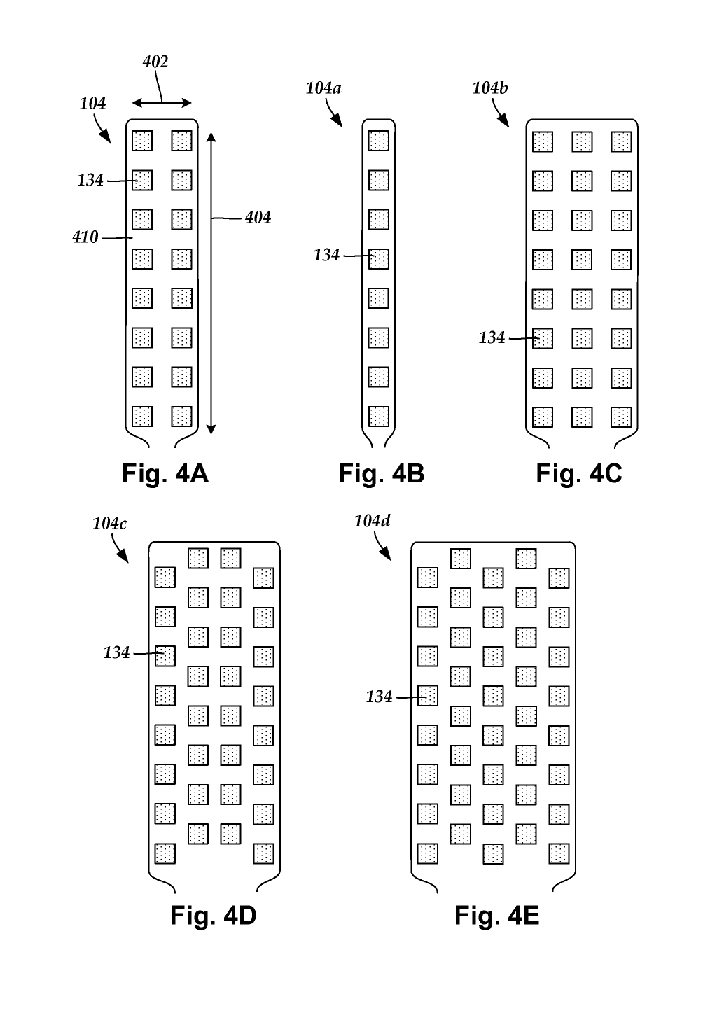

FIGS. 4A-4E show five different electrode configurations, where each electrode configuration has a different number of electrodes per row, but the same number of electrodes (8) per column. The number of electrodes in each column in FIGS. 4A-4E is merely exemplary, and is not meant to be limiting. Other numbers of electrodes can be disposed in a column including, for example, one, two, three, four, five, six, seven, nine, ten, eleven, twelve, thirteen, fourteen, fifteen, sixteen, or more electrodes. Any suitable number of electrodes can also be disposed in a row including, for example, one, two, three, four, five, six, seven, eight, nine, ten, eleven, twelve, thirteen, fourteen, fifteen, sixteen, or more electrodes.

FIG. 4A is a schematic top view of the paddle body 104 having a 2×8 electrode configuration. Accordingly, in FIG. 4A, the paddle body 104 includes two electrodes 134 per row 402, and eight electrodes 134 per column 404. The electrodes 134 are disposed on a first major surface 410. FIG. 4B is a schematic top view of a paddle body 104a having a 1×8 electrode configuration. FIG. 4C is a schematic top view of a paddle body 104b having a 3×8 electrode configuration.

In some embodiments, one or more of the rows or columns can be laterally or longitudinally offset from at least one other row or column. FIG. 4D is a schematic top view of a paddle body 104c having a 4×8 electrode configuration. In FIG. 4D, the two medial columns of electrodes are longitudinally offset from the lateral two columns. FIG. 4E is a schematic top view of a paddle body 104d having a 5×8 electrode configuration.

Patients undergoing electrical stimulation, such as spinal cord stimulation, represent a wide variety of conditions including, for example, chronic pain. A single percutaneous lead or paddle lead may not be able to sufficiently address the patient condition. This may especially be true for patients with disorders where pain may migrate over time, such as complex regional pain syndrome. Accordingly, it may be advantageous to be able to customize stimulation on a patient-by-patient basis.

As herein described, a system and method for customizing the configuration of electrodes implanted into a patient is disclosed. Customizing the electrode configuration can, in turn, customize stimulation. The customizable electrode configurations described herein enable versatility in the amount of electrodes, as well as the physical arrangement of electrodes, used to provide therapy to the patient. Electrodes can be quickly and easily added or removed or, in some cases, moved to a different location. Moreover, customization of the electrode configurations can be performed at the location of the implantation procedure by a medical practitioner. Thus, customization of the electrode configurations can be performed during, or immediately prior, to an implantation procedure.

In some embodiments, one or more leads (e.g., percutaneous leads, paddle leads, or both) can be coupled to a lead scaffold (“scaffold”) that defines a plurality of channels. The one or more leads can be coupled to the scaffold in different numbers, or different arrangements, or both, to customize the configuration of electrodes, thereby potentially customizing the stimulation received by the patient. In some embodiments, the paddle body may include a plurality of connectable stimulation members, where each connectable stimulation member includes at least one electrode. The two or more connectable stimulation members can be coupled together in different numbers, or different arrangements, or both, to customize the electrode configuration, thereby potentially customizing the stimulation received by the patient.

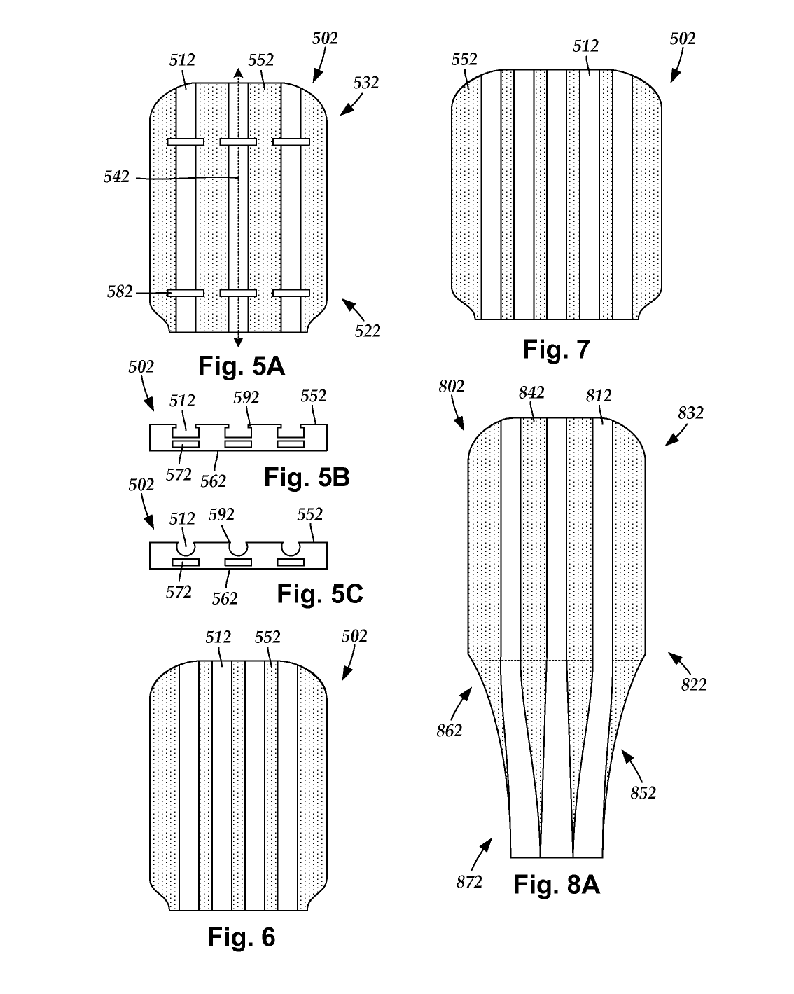

In some embodiments, a scaffold can be used to customize the electrode configuration of the electrical stimulation system. FIG. 5A is a schematic top view of one embodiment of a scaffold 502 defining channels, such as channel 512. The dimensions, or the transverse shapes, of the channels 512 can be adapted to receive specific types of leads. For example, the channels 512 can be configured and arranged to receive one or more of the paddle bodies 104a, or one or more of the percutaneous leads 107′, or a combination thereof.

FIG. 5B is a schematic side view of one embodiment of the scaffold 502 defining channels 512 for receiving one or more of the paddle bodies 104a. In FIG. 5B, the channels 512 have rectangular transverse cross-sections. FIG. 5C is a schematic side view of one embodiment of the scaffold 502 defining channels 512 for receiving one or more of the percutaneous leads 107′. In FIG. 5C, the channels 512 have transverse cross-sections that define at least half of a circle.

The scaffold 502 includes a first end 522, an opposing second end 532, and a longitudinal axis 542. The scaffold 502 is substantially planar and has a first major surface 552 and an opposing second major surface 562. In preferred embodiments, the channels 512 are all defined along the first major surface 552. In alternate embodiments, at least one of the channels 512 is defined along the second major surface 562 and at least one of the channels 512 is defined along the first major surface 552.

The scaffold 502 can include one or more identifiers 572 in proximity to one or more of the channels 512. The one or more identifiers 572 can be disposed at the first end 522, the second end 532, or both. Alternately or additionally, the one or more identifiers 562 can be disposed along the first major surface 552, the second major surface 562, or both.

The one or more identifiers 572 can be used to distinguish one or more of the channels 512 from one or more of the remaining channels 512. For example, the one or more identifiers 572 can be used to label a specific channel 512 for receiving a particular type of lead, or a lead with a particular stimulation pattern, or the like. The one or more identifiers 572 can include, for example, one or more marks, symbols, colors, bar codes, alphanumeric codes, or the like. The one or more identifiers 572 can also be used to facilitate implantation. For example, the one or more identifiers 572 may include one or more radiopaque markers that are detectable by fluoroscopy.

The channels 512 are preferably parallel to one another and extend along the longitudinal axis 542 of the scaffold 502. The channels 512 can extend along either the entire longitudinal axis 542 of the scaffold 502, or a portion thereof. The scaffold 502 can have any suitable lateral center-to-center spacing between adjacent channels 512. In some cases, the center-to-center spacing between adjacent channels 512 is at least 1 mm, 2 mm, 3 mm, 4 mm, or greater. In some cases, the center-to-center spacing between adjacent channels 512 is no greater than 4 mm, 3 mm, 2 mm, 1 mm, or less. In at least some embodiments, the center-to-center spacing between adjacent channels 512 is at least 2 mm and no greater than 3 mm. In some cases, the center-to-center spacing between adjacent channels 512 is the same for each of the channels 512 of the scaffold 502. In other cases, the center-to-center spacing may vary between different adjacent channels 512.

The scaffold 502 can be either rigid or flexible. In some cases, the scaffold 502 may include a combination of one or more rigid regions and one or more comparatively flexible regions. The scaffold 502 can be formed from any material suitable for implantation including, for example, silicone, metal, alloy, polyurethane, PEEK, or the like or combinations thereof. One or more coatings can be applied to the scaffold 502. For example, a hydrophilic coating can be applied to the scaffold 502 to facilitate implantation by increasing the ability of the scaffold 502 to slide along patient tissue, or by increasing the ability of leads (e.g., paddle bodies 104a, percutaneous leads 107′, or the like) to slide into channels 512.

One or more leads can couple to the channels 512 of the scaffold 502 in any suitable manner. For example, the scaffold 502 may be flexible enough to deform, enabling the lead(s) to snap into the channels 512. The scaffold 502 may enable the lead(s) to slide or twist into the channels 512. Preferably, one or more of the channels 512 can removably receive one or more leads. One or more of the channels 512 can, optionally, permanently receive one or more leads.

The scaffold 502 may include one or more retaining features 582 for retaining, or locking, the lead(s) in place. The one or more retaining features 582 can include, for example, one or more tabs 582 (see e.g., FIG. 5A). Optionally, the leads can be retained in the channels 512 by one or more overhanging lips 592 (see e.g., FIG. 5B and FIG. 5C), or the like. The leads can also be retained, at least in part, by an interference fit, one or more adhesives, or the like.

In some cases, a bias feature may be implemented to retain leads in channels 512. For example, a lead may be inserted into one of the channels 512 while a stylet is disposed in the lead, thereby straightening the lead. Removal of the stylet may form a bias in the lead that exerts a force against one or more sides of the channel 512 within which the lead is disposed, thereby retaining the lead within the channel 512.

The scaffold 502 can define any suitable number of channels 512. In FIGS. 5A-5C, the scaffold 502 is shown with three channels 502. FIG. 6 is a schematic top view of another embodiment of the scaffold 502 defining four channels 512. FIG. 7 is a schematic top view of yet another embodiment of the scaffold 502 defining five channels 512. The scaffold may, alternatively, define other numbers of channels 512 including, for example, one, two, six, seven, eight, nine, ten, or more channels 512.

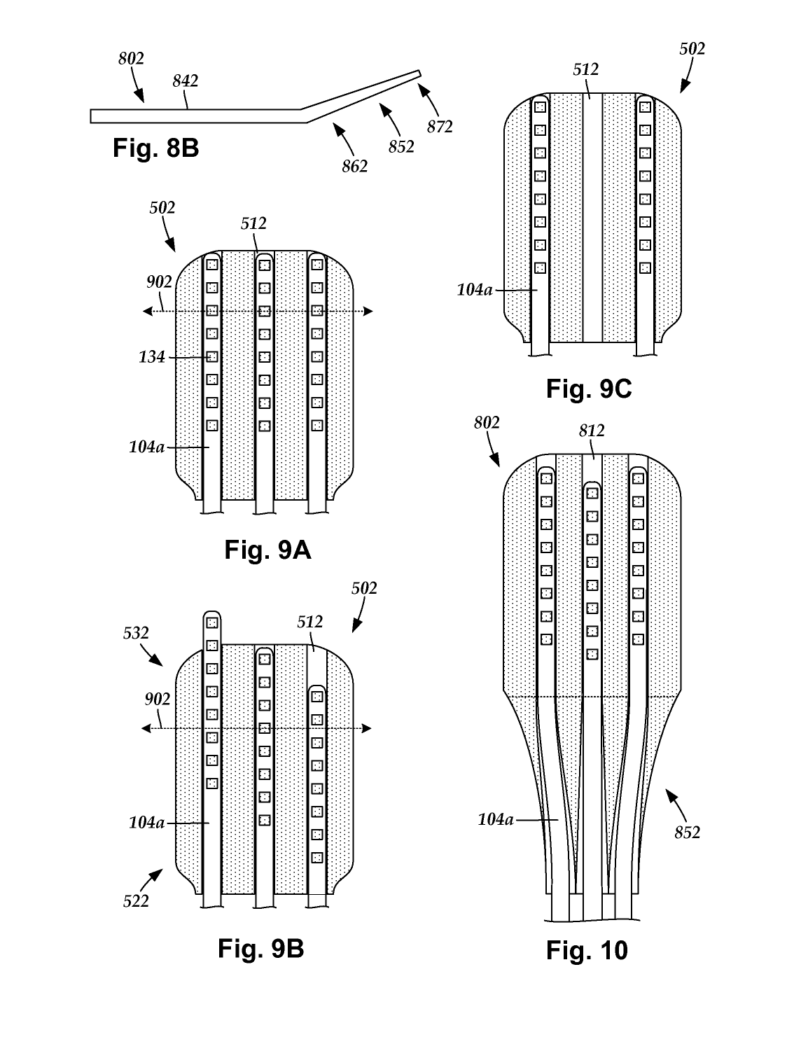

The scaffold can, optionally, include a guide feature for facilitating implantation of the lead(s). FIG. 8A is a schematic top view of yet another embodiment of a scaffold 802 defining channels, such as channel 812, for receiving one or more of the paddle bodies 104a, one or more of the percutaneous leads 107′, or a combination thereof. FIG. 8B is a schematic side view of the scaffold 802. The scaffold 802 has a first end 822, an opposing second end 832, and a first major surface 842 within which the channels 812 are defined.

The scaffold 802 includes a guide feature 852. The guide feature 852 is a tapered extension of the scaffold 802. When the scaffold 802 is implanted, for example, in the patient's epidural space, the scaffold 802 can be inserted into the epidural space such that the guide feature 852 extends from the epidural space, thereby providing a visual cue for a medical practitioner to use for guiding the scaffold 802 within the epidural space. In some cases, the guide feature 852 can be formed as part of the scaffold 802. In other cases, the guide feature 852 can be coupled to the scaffold 802.

The guide feature 852 includes a connection end 862 and an opposing guiding end 872. The connection end 862 couples to the first end 822 of the scaffold 802. The connection end 862 can have a width that is equal to a width of the lead scaffold 802 at the first end 822. The guiding end 872 of the guide feature 852 extends away from the first end 822 of the lead scaffold 802. The guide feature 852 tapers such that the connection end 862 has a diameter that is greater than a diameter of the guiding end 872. In alternate embodiments, the guide feature 852 tapers such that that the guiding end 872 has a diameter that is greater than a diameter of the connection end 862.

Optionally, the guide feature 852 can be angled with respect to the first major surface 842. In FIG. 8B, the guide feature 852 is shown angled upward, toward the first major surface 842. Alternately, the guide feature 852 can be angled downward, away from the first major surface 842. In some cases, the channels 812 may extend along the guide feature 852. Alternately, the guide feature 852 may define one or more lumens in lieu of channels.

When the channels 812 (or lumens) extend along the guide features 852, the channels 812 (or lumens) may bend with the tapering of the guide feature 852. Consequently, the lateral center-to-center spacing between adjacent channels 812 (or lumens) may be reduced along the guide feature 852. In some cases, the channels 812 (or lumens) may be tapered from the guiding end 872 to the connection end 862 to funnel inserted leads into the channels 812 extending along the scaffold 802 (see e.g., FIG. 8A and FIG. 10).

The paddle bodies 104a (or the distal ends of the percutaneous leads 107′) can be disposed in the channels such that the electrodes 134 are either inset from, flush with, or protruding from the first major surface 552, 842 of the scaffold. The paddle bodies 104a (or the distal ends of the percutaneous leads 107′) can also be disposed in the channels such that the distal ends of the leads are either extended axially from the second end 532, 832 of the scaffold, flush with the second end 532, 832 of the scaffold, or disposed somewhere between the first end 522, 822 and the second end 532, 832 of the scaffold. In some cases, the paddle bodies 104a (or the distal ends of the percutaneous leads 107′) may be prevented from extending axially beyond the second end 532, 832 of the scaffold (e.g., the second end 532, 832 of the scaffold may physically obstruct one or more of the channels 512, 812).

FIGS. 9A-9C show three different exemplary electrode configurations that can be formed using two or more 1×8 paddle bodies 104a. FIG. 9A is a schematic top view of one embodiment of a plurality of the paddle bodies 104a disposed on the scaffold 502. In FIG. 9A, one of the paddle bodies 104a is disposed in each of the channels 512 of the scaffold 502. The paddle bodies 104a are disposed in the channels 512 such that at least one electrode 134 of each of the paddle bodies 104a align along an axis 902 that is transverse to the longitudinal axis (542 in FIG. 5A). The paddle bodies 104a are also disposed in the channels 512 such that the distal ends of the paddle bodies 104a are flush with the second end 532 of the scaffold 502. Alternately, the paddle bodies 104a can be disposed in the channels 512 such that the electrodes 134 are aligned along the axis 902 and the distal ends of the paddle bodies 104a are either extended axially from the second end 532 of the scaffold 502 or disposed somewhere between the first end 522 and the second end 532 of the scaffold 502.

FIG. 9B is a schematic top view of one embodiment of a plurality of the paddle bodies 104a disposed on the scaffold 502. In FIG. 9B, one of the paddle bodies 104a is disposed in each of the channels 512 of the scaffold 502. The paddle bodies 104a are disposed in the channels 512 such that at least one electrode 134 of at least one of the paddle bodies 104a is longitudinally offset from at least one other electrode 134 of at least one other of the paddle bodies 104a along the transverse axis 902. In FIG. 9B, one of the paddle bodies 104a is shown extending axially beyond the second end 532. In some cases, one or more of the paddle bodies 104a can be disposed on the scaffold 502 such that one or more of the electrodes 134 can extend axially from either (or both) the first end 522 or the second end 532 of the scaffold 502. In some cases, the paddle bodies 104a can be disposed in the channels with longitudinally offset electrodes such that the distal ends of each of the paddle bodies 104a are disposed between the first end 522 and the second end 532.

The scaffold can, optionally, be implanted in a patient without a lead being disposed in each of the channels. FIG. 9C is a schematic top view of one embodiment of a plurality of the paddle bodies 104a disposed on the scaffold 502. In FIG. 9C, there is no lead disposed in a middle channel 512 of the scaffold. Consequently, the lateral center-to-center spacing between adjacent electrodes 134 is between the two outer channels, instead of between one of the outer channels and the middle channel. Thus, the lateral center-to-center spacing between adjacent electrodes 134 is increased when the lead bodies 104a are disposed in the outer channels and the middle channel is left empty. Note that different channels can be left empty (e.g., one or both of the outer channels). Note also that, when the scaffold includes more than three channels, a larger number of lateral-spacing variations are obtainable by not disposing a lead in one or more of the channels.

FIG. 10 is a schematic top view of one embodiment of a plurality of the paddle bodies 104a disposed on the scaffold 802. When leads are disposed on the scaffold 802, the tapering guide element 852 may cause at least one of the leads to bend with a corresponding channel 812. In FIG. 10, the outer channels bend towards the middle channel along the guide element 852. In preferred embodiments, the lead bodies 104a are disposed in the channels 812 such that the bent portions of the channels 812 are disposed proximal to the electrodes 134 of the lead bodies 104a (or proximal to the electrodes 134 of the percutaneous leads 107′).

In some embodiments, a plurality of connectable stimulation members can be used to customize the electrode configuration of the electrical stimulation system. The plurality of connectable stimulation members can be coupled together in different numbers, or different arrangements, to form a lead body (e.g., a paddle body). Optionally, one or more non-conductive spacers can be disposed between two or more connectable stimulation members.

The connectable stimulation members can, optionally, be formed such that each of the connectable stimulation members includes at least one electrode disposed on a member body and a lead body extending from the member body. Any suitable number of electrodes can be disposed on the member body including, for example, one, two, three, four, five, six, seven, eight, or more electrodes. In some cases, the same number of electrodes is disposed on each of the connectable stimulation members. In other cases, a different number of electrodes is disposed on at least one of the connectable stimulation members from at least one other of the connectable stimulation members.

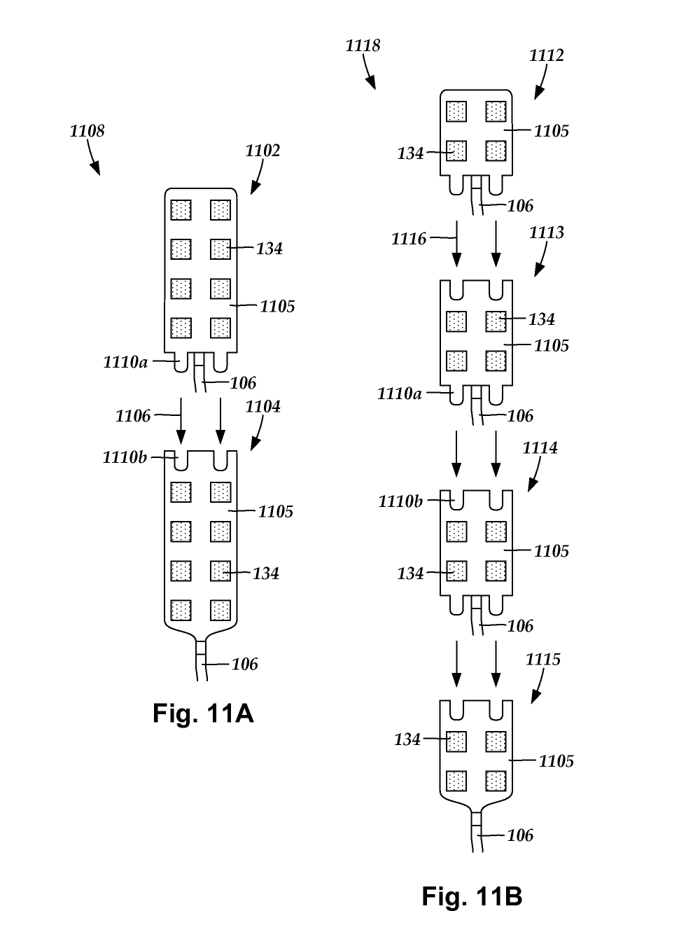

FIG. 11A is a schematic top view of one embodiment of connectable stimulation members 1102 and 1104 each having a 2×4 electrode configuration. The connectable stimulation members 1102 and 1104 are axially coupleable, as shown by arrows 1106, to form a paddle body 1108 with a 2×8 electrode configuration (see e.g., paddle body 104). The connectable stimulation members 1102 and 1104 each include a member body 1105.

For each of the connectable stimulation members 1102 and 1104, at least one lead body 106 is coupled to the member body 1105 and is configured and arranged to electrically couple the electrodes 134 of the connectable stimulation member to the control module (102 in FIG. 1). When multiple lead bodies 106 are used, the lead bodies 106 can be coupled either to the same control module, or to different control modules. In some cases, the electrodes 134 of the connectable stimulation members 1102 and 1104 may transmit different stimulation patterns or intensities from one another.

The member bodies 1105 include one or more mechanical coupling elements (“coupling elements”) 1110a and 1110b, respectively. The coupling elements 1110a, 1110b can be disposed on one or more edges of the member bodies 1105. Any suitable coupling element 1110a, 1110b can be used including, for example, matable features (e.g., corresponding slots and tabs, corresponding male and female features), or the like. The coupling elements 1110a, 1110b can include one or more interlocking features. The coupling elements 1110a, 1110b can include one or more fastening members (e.g., snaps, clips, or the like). In some cases, one or more of the coupling elements 1110a, 1110b can be formed from a shape memory material that changes shape when the connectable stimulation members 1102 and 1104 are exposed to body temperature, the change in shape causing the coupling elements 1110a, 1110b to couple together. In some cases, the coupling elements 1110a, 1110b can be coupled together using a tool (e.g., a wrench, a screwdriver, or the like). In FIGS. 11A-14, the coupling element 1110a is shown as a male feature and the coupling element 1110b is shown as a corresponding female feature. In some instances, adhesive may be used to facilitate coupling.

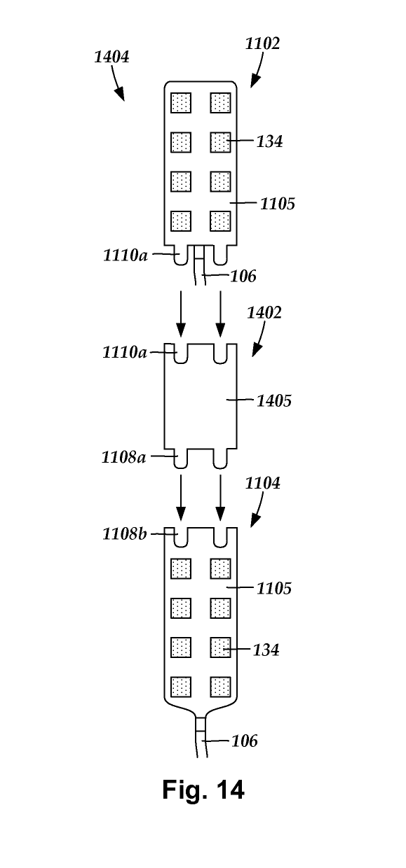

A member body 1105 of a given connectable stimulation member can include one or more of either, or both, coupling element 1110a or 1110b. Note that the coupling elements 1110a and 1110b can be used with any of the member bodies 1105 shown or discussed, with reference to FIGS. 11A-14. Note also that the coupling elements 1110a and 1110b can be used with any spacers, as well (see FIG. 14).

The member bodies 1105 can be either rigid or flexible. In some cases, one of the member bodies 1105 may be more flexible than the other. For example, the member body 1105 of the connectable stimulation member 1102 can be relatively rigid or flexible when compared to the member body 1105 of the connectable stimulation member 1104. The member bodies 1105 can be formed from any material suitable for implantation including, for example, silicone, metal, alloy, polyurethane, PEEK, or the like or combinations thereof. One or more coatings can be applied to the member bodies 1105. For example, a hydrophilic coating can be applied to the member bodies 1105 to facilitate implantation by increasing the ability of the connectable stimulation members 1102 and 1104 to slide along patient tissue. Note that the above-mentioned materials and coatings can be used by any of the member bodies (or spacers) shown or discussed, with reference to FIGS. 11A-14.

In FIG. 11A, the paddle lead 1108 is formed from two connectable stimulation members 1102 and 1104 that couple together axially with one another. The paddle lead 1108 can be formed from any suitable number of axially-connectable stimulation members including, for example, two, three, four, five, six, seven, eight, or more connectable stimulation members. FIG. 11B is a schematic top view of one embodiment of a plurality of connectable stimulation members 1112-1115. Each of the connectable stimulation members 1112-1115 has a 2×2 electrode configuration. The connectable stimulation members 1112-1115 are axially coupleable, as shown by arrows 1116, to form a paddle body 1118 with a 2×8 electrode configuration (see e.g., paddle body 104 of FIG. 1).

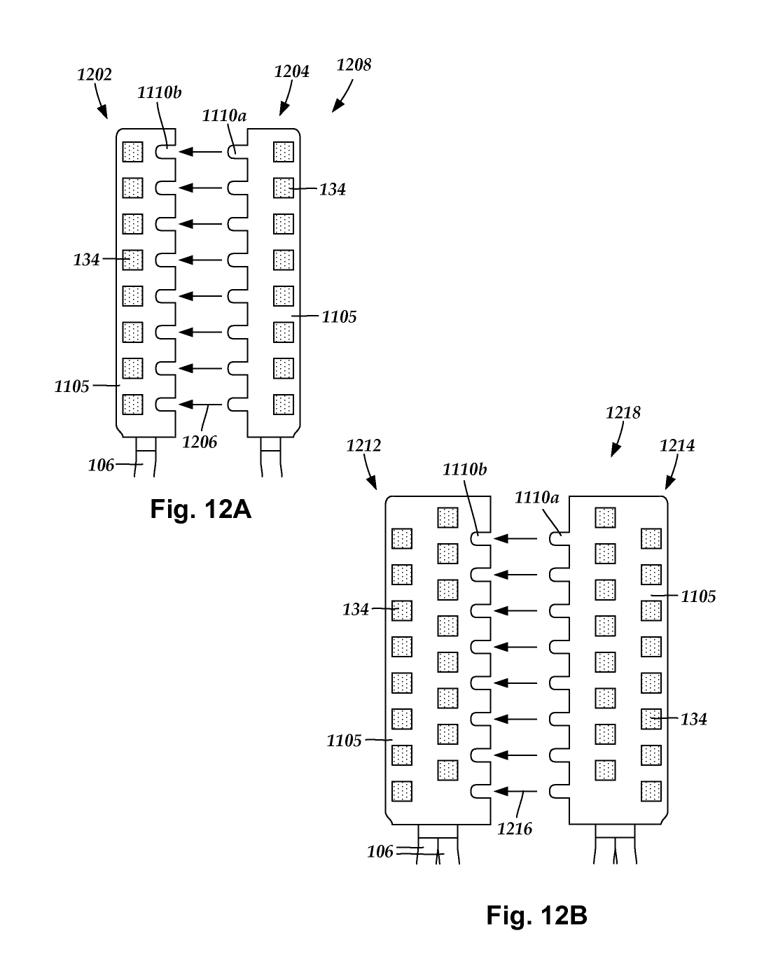

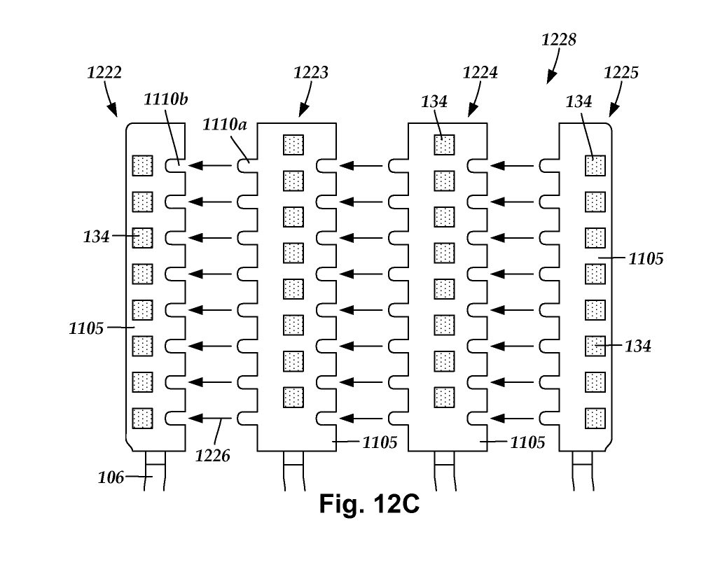

In at least some embodiments, the connectable stimulation members can couple together laterally in lieu of the axially coupling shown in FIGS. 11A-11B. FIG. 12A is a schematic top view of one embodiment of a plurality of connectable stimulation members 1202 and 1204. Each of the connectable stimulation members 1202 and 1204 has a 1×8 electrode configuration and couple together laterally, as shown by arrows 1206. The connectable stimulation members 1202 and 1204 are combinable to form a paddle body 1208 with a 2×8 electrode configuration (see e.g., paddle body 104 of FIG. 1).

FIG. 12B is a schematic top view of one embodiment of a plurality of connectable stimulation members 1212 and 1214. Each of the connectable stimulation members 1212 and 1214 has a 2×8 electrode configuration and couple together laterally, as shown by arrows 1216. The connectable stimulation members 1212 and 1214 are combinable to form a paddle body 1218 with a 4×8 electrode configuration (see e.g., paddle body 104d of FIG. 4D).

FIG. 12C is a schematic top view of one embodiment of a plurality of connectable stimulation members 1222-1225. Each of the connectable stimulation members 1222-1225 has a 1×8 electrode configuration and couple together laterally, as shown by arrows 1226. The connectable stimulation members 1222-1225 are combinable to form a paddle body 1228 with a 4×8 electrode configuration (see e.g., paddle body 104d of FIG. 4D).

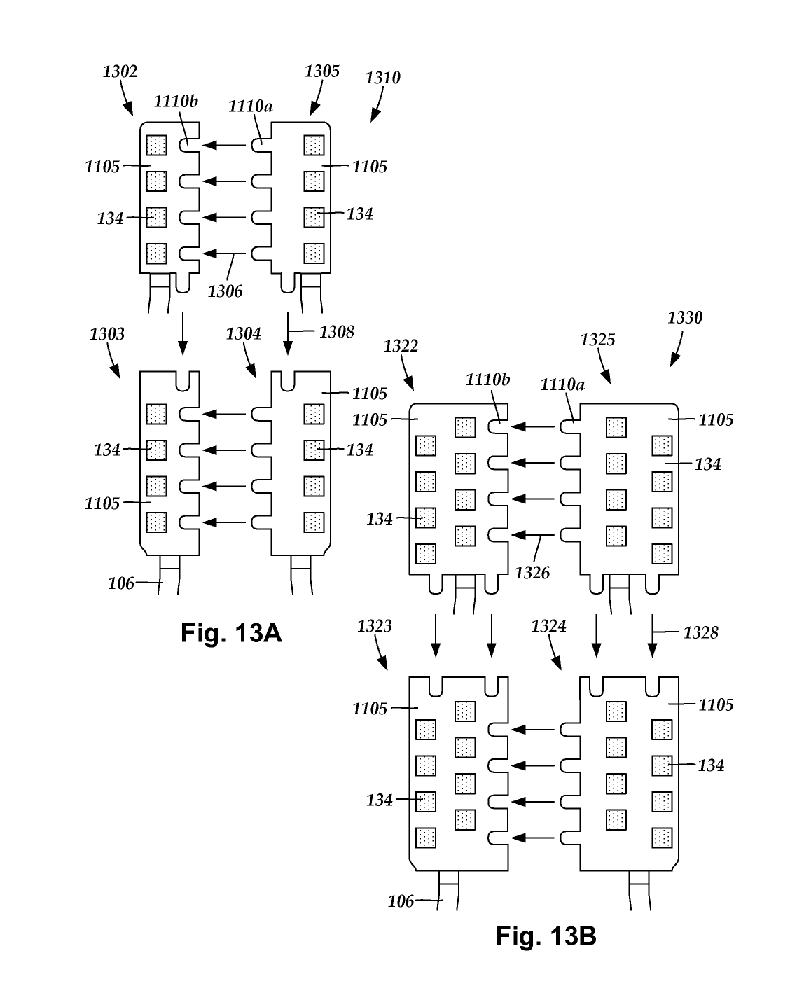

In at least some embodiments, the connectable stimulation members can couple together laterally and axially. FIG. 13A is a schematic top view of one embodiment of a plurality of connectable stimulation members 1302-1305. Each of the connectable stimulation members 1302-1305 has a 1×4 electrode configuration and couple together both laterally, as shown by lateral arrows 1306, and axially, as shown by axial arrows 1308. The connectable stimulation members 1302-1305 are combinable to form a paddle body 1310 with a 2×8 electrode configuration (see e.g., paddle body 104 of FIG. 1).

FIG. 13B is a schematic top view of one embodiment of a plurality of connectable stimulation members 1322-1325. Each of the connectable stimulation members 1322-1325 has a 2×4 electrode configuration and couple together both laterally, as shown by lateral arrows 1326, and axially, as shown by axial arrows 1328. The connectable stimulation members 1322-1325 are combinable to form a paddle body 1330 with a 4×8 electrode configuration (see e.g., paddle body 104d of FIG. 4D).

Any suitable number of connectable stimulation members can be coupled together to form the paddle body. Any suitable number of electrodes can be disposed on each of the connectable stimulation members. When multiple electrodes are disposed on a given connectable stimulation member, the electrodes can be arranged in any suitable arrangement (e.g., rows and columns, or the like). In FIGS. 11A-14, the electrodes are shown arranged into rows and columns. The number of electrodes 134 in each column in FIGS. 11A-14 is merely exemplary, and is not meant to be limiting. Other numbers of electrodes 134 can be disposed in a column including, for example, one, two, three, four, five, six, seven, eight, nine, ten, eleven, twelve, thirteen, fourteen, fifteen, sixteen, or more electrodes 134. Any suitable number of electrodes 134 can also be disposed in a row including, for example, one, two, three, four, five, six, seven, eight, nine, ten, eleven, twelve, thirteen, fourteen, fifteen, sixteen, or more electrodes 134.

Optionally, one or more spacers can be disposed between two or more connectable stimulation members. FIG. 14 is a schematic top view of one embodiment of a spacer 1402 disposed between the connectable stimulation members 1102 and 1104 (see FIG. 11A). The spacer 1402 can be used to increase the distance between the electrodes 134 of the connectable stimulation member 1102 from the electrodes 134 of the connectable stimulation member 1104. The spacer 1402 and the connectable stimulation members 1102 and 1104 can be combined to form a paddle body 1404.

The spacer 1402 includes a spacer body 1405 that is coupleable with other member bodies 1402 (e.g., or other spacers) in the same way as connectable stimulation members couple to one another. The spacers 1402 may, for example, include one or more coupling elements 1110a or 1110b that mate with corresponding coupling elements 1110a and 1110b disposed on flanking connectable stimulation members. In some cases, the spacers 1402 are similar to the connectable stimulation members in size and shape. In other cases, the spacers 1402 are smaller or larger than flanking connectable stimulation members along at least one axis. Any suitable number of spacers 1402 can be used in conjunction with the connectable stimulation members to create a desired distance between electrodes 134 of flanking connectable stimulation members.

The spacers bodies 1405 can have the same rigidity as the member bodies 1105, or the spacer bodies 1405 can be either more or less rigid than the member bodies 1105. The spacers bodies 1405 can be formed from any material suitable for implantation including, for example, silicone, polyurethane, PEEK, metal, alloy, or the like or combinations thereof. One or more coatings can be applied to the spacer bodies 1405. For example, a hydrophilic coating can be applied to the spacers bodies 1405 to facilitate implantation by increasing the ability of the spacer 1402 to slide along patient tissue.

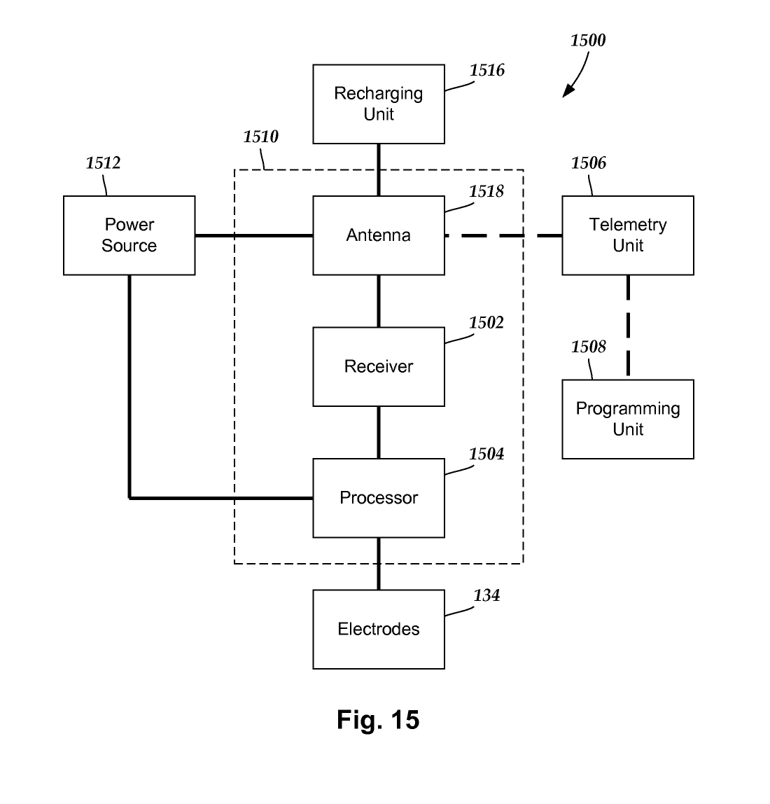

FIG. 15 is a schematic overview of one embodiment of components of an electrical stimulation system 1500 including an electronic subassembly 1510 disposed within a control module. It will be understood that the electrical stimulation system can include more, fewer, or different components and can have a variety of different configurations including those configurations disclosed in the stimulator references cited herein.

Some of the components (for example, power source 1512, antenna 1518, receiver 1502, and processor 1504) of the electrical stimulation system can be positioned on one or more circuit boards or similar carriers within a sealed housing of an implantable pulse generator, if desired. Any power source 1512 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Patent Application Publication No. 2004/0059392, incorporated herein by reference.

As another alternative, power can be supplied by an external power source through inductive coupling via the optional antenna 1518 or a secondary antenna. The external power source can be in a device that is mounted on the skin of the user or in a unit that is provided near the user on a permanent or periodic basis.

If the power source 1512 is a rechargeable battery, the battery may be recharged using the optional antenna 1518, if desired. Power can be provided to the battery for recharging by inductively coupling the battery through the antenna to a recharging unit 1516 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. A processor 1504 is generally included to control the timing and electrical characteristics of the electrical stimulation system. For example, the processor 1504 can, if desired, control one or more of the timing, frequency, strength, duration, and waveform of the pulses. In addition, the processor 1504 can select which electrodes can be used to provide stimulation, if desired. In some embodiments, the processor 1504 may select which electrode(s) are cathodes and which electrode(s) are anodes. In some embodiments, the processor 1504 may be used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 1508 that, for example, allows modification of pulse characteristics. In the illustrated embodiment, the processor 1504 is coupled to a receiver 1502 which, in turn, is coupled to the optional antenna 1518. This allows the processor 1504 to receive instructions from an external source to, for example, direct the pulse characteristics and the selection of electrodes, if desired.

In one embodiment, the antenna 1518 is capable of receiving signals (e.g., RF signals) from an external telemetry unit 1506 which is programmed by a programming unit 1508. The programming unit 1508 can be external to, or part of, the telemetry unit 1506. The telemetry unit 1506 can be a device that is worn on the skin of the user or can be carried by the user and can have a form similar to a pager, cellular phone, or remote control, if desired. As another alternative, the telemetry unit 1506 may not be worn or carried by the user but may only be available at a home station or at a clinician's office. The programming unit 1508 can be any unit that can provide information to the telemetry unit 1506 for transmission to the electrical stimulation system 1500. The programming unit 1508 can be part of the telemetry unit 1506 or can provide signals or information to the telemetry unit 1506 via a wireless or wired connection. One example of a suitable programming unit is a computer operated by the user or clinician to send signals to the telemetry unit 1506.

The signals sent to the processor 1504 via the antenna 1518 and receiver 1502 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 1500 to cease operation, to start operation, to start charging the battery, or to stop charging the battery. In other embodiments, the stimulation system does not include an antenna 1518 or receiver 1502 and the processor 1504 operates as programmed.

Optionally, the electrical stimulation system 1500 may include a transmitter (not shown) coupled to the processor 1504 and the antenna 1518 for transmitting signals back to the telemetry unit 1506 or another unit capable of receiving the signals. For example, the electrical stimulation system 1500 may transmit signals indicating whether the electrical stimulation system 1500 is operating properly or not or indicating when the battery needs to be charged or the level of charge remaining in the battery. The processor 1504 may also be capable of transmitting information about the pulse characteristics so that a user or clinician can determine or verify the characteristics.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A lead assembly for an electrical stimulation system, the lead assembly comprising:
a substantially planar lead scaffold having a first end, an opposing second end, a longitudinal length, a first major surface, and an opposing second major surface, the lead scaffold defining a first channel, a second channel, and a third channel each defined along the first major surface from the first end of the lead scaffold, wherein the first, second, and third channels are parallel to one another and to the longitudinal length of the lead scaffold;
a guide feature coupled to the first end of the lead scaffold, the guide feature having a connection end, an opposing guiding end, and a longitudinal length, the connection end being coupled to the lead scaffold, wherein the guide feature is tapered along its longitudinal length such that the connection end has a diameter that is greater than a diameter of the guiding end; and
a first lead and a second lead, wherein the first lead and the second lead each have a proximal end and an opposing distal end, and wherein the first lead and the second lead each comprise
a plurality of electrodes disposed at the distal end of the lead,
a plurality of terminals disposed at the proximal end of the lead, and
a plurality of conductive wires coupling the plurality of electrodes electrically to the plurality of terminals;
wherein the first lead is insertable into the first channel such that at least one of the electrodes of the first lead is disposed in the first channel between the first end and the second end of the lead scaffold;
wherein the second lead is insertable into the second channel such that at least one of the electrodes of the second lead is disposed in the second channel between the first end and the second end of the lead scaffold.

2. The lead assembly of claim 1, wherein the guide feature defines a first channel, a second channel, and a third channel, and wherein the first, second, and third channels of the guide feature are aligned with the corresponding first, second, and third channels of the lead scaffold.

3. The lead assembly of claim 1, wherein at least one electrode of the first lead is aligned with at least one electrode of the second lead along at least one axis perpendicular to the longitudinal length of the lead scaffold.

4. The lead assembly of claim 1, wherein at least one electrode of the first lead is longitudinally offset from at least one electrode of the second lead along at least one axis perpendicular to the longitudinal length of the lead scaffold.

5. The lead assembly of claim 1, wherein the first, second, and third channels of the lead scaffold extend along the first major surface such that the second channel extends between the first channel and the third channel.

6. The lead assembly of claim 1, wherein the first, second, and third channels of the lead scaffold extend along the first major surface such that the third channel extends between the first channel and the second channel.

7. The lead assembly of claim 1, wherein the first lead is a paddle lead having a single column of electrodes.

8. An electrical stimulation kit comprising:
a plurality of connectable stimulation members configured and arranged for coupling together with one another, each of the connectable stimulation members comprising
a member body,
at least one electrode disposed on the member body,
a mechanical coupling element disposed on an edge of the member body, the mechanical coupling element configured and arranged to mechanically couple with a corresponding mechanical coupling element of another one of the plurality of connectable stimulation members,
at least one lead body coupled to the member body,
at least one terminal disposed on each of the at least one lead bodies, and
a plurality of conductive wires coupling the at least one electrode electrically to the at least one terminal.

9. The kit of claim 8, further comprising at least one spacer configured and arranged to couple with at least one of the plurality of connectable stimulation members.

10. The kit of claim 9, wherein each of the at least one spacer comprises a spacer body and a mechanical coupling element disposed on each of at least two edges of the spacer body, each of the mechanical coupling elements configured and arranged to mechanically couple with a corresponding mechanical coupling element of either another spacer body or one of the connectable stimulation members.

11. The kit of claim 8, wherein the mechanical coupling element comprises at least one of a male feature or a corresponding female feature and the corresponding mechanical coupling element of another one of the plurality of connectable stimulation members comprises the other of the least one male feature or corresponding female feature.

12. The kit of claim 8, wherein the mechanical coupling element is formed from a shape memory material.

13. A lead for an electrical stimulation system, the lead comprising the kit of claim 8;
wherein the mechanical coupling element of one of the plurality of connectable stimulation members of the kit is coupled to the corresponding mechanical coupling element of at least one other of the plurality of connectable stimulation members.

14. A lead for an electrical stimulation system, the lead comprising:
a plurality of connectable stimulation members coupled together with one another, each of the connectable stimulation members comprising
a member body,
at least one electrode disposed on the member body,
at least mechanical coupling element disposed on an edge of the paddle body, the at least one coupling element configured and arranged to mechanically couple with a corresponding mechanical coupling element of another one of the connectable stimulation members,
at least one lead body coupled to the member body,
at least one terminal disposed on each of the at least one lead bodies, and
a plurality of conductive wires coupling the at least one electrode electrically to the at least one terminal.

15. A method of customizing stimulation by an implantable stimulation system, the method comprising;
providing a substantially planar lead scaffold having a first end, an opposing second end, a longitudinal length, a first major surface, and an opposing second major surface, the lead scaffold defining a first channel, a second channel, and a third channel each defined along the first major surface from the first end of the lead scaffold, wherein the first, second, and third channels are each parallel to one another and extend along axes parallel to the longitudinal length;
coupling a guide feature to the first end of the lead scaffold, the guide feature having a connection end, an opposing guiding end, and a longitudinal length, the connection end being coupled to the lead scaffold, wherein the guide feature is tapered along its longitudinal length such that the connection end has a diameter that is greater than a diameter of the guiding end;
inserting a first lead into the first channel of the lead scaffold, wherein the first lead has a proximal end and an opposing distal end, and wherein the first lead comprises a plurality of electrodes disposed at the distal end of the first lead, a plurality of terminals disposed at the proximal end of the first lead, and a plurality of conductive wires coupling the plurality of electrodes electrically to the plurality of terminals;
inserting a second lead into the second channel of the lead scaffold, wherein the second lead has a proximal end and an opposing distal end, and wherein the second lead comprises a plurality of electrodes disposed at the distal end of the second lead, a plurality of terminals disposed at the proximal end of the second lead, and a plurality of conductive wires coupling the plurality of electrodes electrically to the plurality of terminals; and
implanting the lead scaffold into a patient.

16. The method of claim 15, wherein providing the lead scaffold defining the first channel, the second channel, and the third channel each defined along the first major surface comprises providing the lead scaffold defining the first, second, and third channels, wherein the second channel extends between the first channel and the third channel.

17. The method of claim 15, wherein providing the lead scaffold defining the first channel, the second channel, and the third channel each defined along the first major surface comprises providing the lead scaffold defining the first, second, and third channels, wherein the third channel extends between the first channel and the second channel.

18. The method of claim 15, wherein inserting the first lead into the first channel of the lead scaffold comprises inserting the first lead into the first channel of the lead scaffold such that at least a portion of the distal end of the first lead extends axially from the second end of the lead scaffold.

19. The method of claim 15, wherein inserting the second lead into the second channel of the lead scaffold comprises inserting the second lead into the second channel of the lead scaffold such that at least one electrode of the first lead is aligned with at least one electrode of the second lead along at least one axis perpendicular to the longitudinal length of the lead scaffold.

20. The method of claim 15, wherein inserting the second lead into the second channel of the lead scaffold comprises inserting the second lead into the second channel of the lead scaffold such that at least one electrode of the first lead is longitudinally offset from at least one electrode of the second lead along at least one axis perpendicular to the longitudinal length of the lead scaffold.
